SEQUENCE LISTING (31 sequences)

SEQ ID NO: 1 - protein, 9 aa, Homo sapiens
KVLEYVIKV

SEQ ID NO: 2 - protein, 9 aa, Homo sapiens
LQLVFGIDV

SEQ ID NO: 3 - protein, 9 aa, Homo sapiens
SAYGEPRKL

SEQ ID NO: 4 - protein, 9 aa, Homo sapiens
CLGLSYDGL

SEQ ID NO: 5 - protein, 9 aa, Homo sapiens
FLWGPRALA

SEQ ID NO: 6 - protein, 9 aa, Homo sapiens
CILESLFRA

SEQ ID NO: 7 - protein, 9 aa, Homo sapiens
FLIIVLVMI

SEQ ID NO: 8 - protein, 9 aa, Homo sapiens
IMPKTGFLI

SEQ ID NO: 9 - protein, 9 aa, Homo sapiens
KVADLVGFL

SEQ ID NO: 10 - protein, 9 aa, Homo sapiens
LVLGTLEEV

SEQ ID NO: 11 - protein, 9 aa, Homo sapiens
KASESLQLV

SEQ ID NO: 12 - protein, 9 aa, Homo sapiens
YVLVTCLGL

SEQ ID NO: 13 - protein, 9 aa, Homo sapiens
YVIKVSARV

SEQ ID NO: 14 - protein, 9 aa, Homo sapiens
RALAETSYV

SEQ ID NO: 15 - protein, 9 aa, Homo sapiens
VITKKVADL

SEQ ID NO: 16 - protein, 9 aa, Homo sapiens
LIIVLVMIA

SEQ ID NO: 17 - protein, 9 aa, Homo sapiens
GTLEEVPTA

SEQ ID NO: 18 - protein, 9 aa, Homo sapiens
QEALGLVCV

SEQ ID NO: 19 - protein, 9 aa, Homo sapiens
STSCILESL

SEQ ID NO: 20 - protein, 9 aa, Homo sapiens
TKAEMLESV

SEQ ID NO: 21 - protein, 9 aa, Homo sapiens
KTGFLIIVL

SEQ ID NO: 22 - protein, 9 aa, Homo sapiens
LAETSYVKV

SEQ ID NO: 23 - protein, 9 aa, Homo sapiens
SLHCKPEEA

SEQ ID NO: 24 - protein, 8 aa, Homo sapiens
MSLEQRSL

SEQ ID NO: 25 - protein, 9 aa, Homo sapiens
ALEAQQEAL

SEQ ID NO: 26 - protein, 9 aa, Homo sapiens
ILESLFRAV

SEQ ID NO: 27 - protein, 9 aa, Homo sapiens
ALREEEEGV

SEQ ID NO: 28 - DNA, 27 bp, Homo sapiens
aaagtccttgagtatgtgatcaaggtc

SEQ ID NO: 29 - DNA, 27 bp, Homo sapiens
ttgcagctggtctttggcattgacgtg

SEQ ID NO: 30 - DNA, 27 bp, Homo sapiens
agtgcctatggggagcccaggaagctg

SEQ ID NO: 31 - DNA, 27 bp, Homo sapiens
tgcctaggtctctcctatgatggcctg

(19) United States
(12) Patent Application Publication
(10) Pub. No.: US 2003/0148973 A1
(43) Pub. Date: Aug. 7, 2003

(54) MAGE-A1 peptides for treating or preventing cancer

(76) Inventors: Peter Emtage, Boston, MA (US); Liza Karunakaran, Toronto (CA); Arthur Pedyczak, Toronto (CA); Brian H. Barber, Hawthorne, NY (US)

Correspondence Address: Patrick J. Halloran, Aventis Pasteur, Inc., One Discovery Drive, Swiftwater, PA, 18370, US

(21) Appl. No.: 10/150,797

(22) Filed: May 17, 2002

Related U.S. Application Data
(60) Provisional application No. 60/292,590, filed on May 23, 2001.

(51) Int. Cl. C07H 21/04; A61K 48/00; A61K 39/00

(52) U.S. Cl. 514/44; 424/93.2; 424/185.1; 536/23.1

(57) ABSTRACT

The present invention relates to a nucleic acid encoding a polypeptide and the use of the nucleic acid or polypeptide in preventing and/or treating cancer. In particular, the invention relates to improved vectors for the insertion and expression of foreign genes encoding tumor antigens for use in immunotherapeutic treatment of cancer.

### RELATED APPLICATIONS

This application claims priority to U.S. Ser. No. 60/292,590 filed May 23, 2001.

### FIELD OF THE INVENTION

The present invention relates to a nucleic acid encoding a polypeptide and the use of the nucleic acid or polypeptide in preventing and/or treating cancer. In particular, the invention relates to improved vectors for the insertion and expression of foreign genes encoding tumor antigens for use in immunotherapeutic treatment of cancer.

### BACKGROUND OF THE INVENTION

There has been tremendous increase in last few years in the development of cancer vaccines with Tumour-associated antigens (TAAs) due to the great advances in identification of molecules based on the expression profiling on primary tumours and normal cells with the help of several techniques such as high density microarray, SEREX, immunohistochemistry (IHC), RT-PCR, in-situ hybridization (ISH) and laser capture microscopy (Rosenberg, Immunity, 1999; Sgroi et al, 1999, Schena et al, 1995, Offringa et al, 2000). The TAAs are antigens expressed or over-expressed by tumour cells and could be specific to one or several tumors for example CEA antigen is expressed in colorectal, breast and lung cancers. Sgroi et al (1999) identified several genes differentially expressed in invasive and metastatic carcinoma cells with combined use of laser capture microdissection and cDNA microarrays. Several delivery systems like DNA or viruses could be used for therapeutic vaccination against human cancers (Bonnet et al, 2000) and can elicit immune responses and also break immune tolerance against TAAs. Tumour cells can be rendered more immunogenic by inserting transgenes encoding T cell co-stimulatory molecules such as B7.1 or cytokines such as IFN-γ, IL2, or GM-CSF, among others. Co-expression of a TAA and a cytokine or a co-stimulatory molecule can develop effective therapeutic vaccine (Hodge et al, 95, Bronte et al, 1995, Chamberlain et al, 1996).

There is a need in the art for reagents and methodologies useful in stimulating an immune response to prevent or treat cancers. The present invention provides such reagents and methodologies which overcome many of the difficulties encountered by others in attempting to treat cancers such as cancer.

### SUMMARY OF THE INVENTION

The present invention provides a TA for administration to a patient to prevent and/or treat cancer. In one embodiment, the TA is an immunogenic peptide derived from the sequence of MAGE-1. In preferred embodiments, the TA is a MAGE-1 peptide as shown in SEQ ID NO.: 1, SEQ ID NO.:2, SEQ ID NO.:3, or SEQ ID NO. 4. In certain embodiments, the TA is administered to a patient as a peptide, as part of a polypeptide, or as a nucleic acid encoding the peptide sequence within a plasmid or other delivery vector, such as a recombinant virus. The TA may also be administered in combination with one or more other TAs, angiogenesis-associated antigens (“AA”), and/or co-stimulatory components.

### DETAILED DESCRIPTION

The present invention provides reagents and methodologies useful for treating and/or preventing cancer. All references cited within this application are incorporated by reference.

In one embodiment, the present invention relates to the induction or enhancement of an immune response against one or more tumor antigens (“TA”) to prevent and/or treat cancer. In certain embodiments, one or more TAs may be combined. In preferred embodiments, the immune response results from expression of a TA in a host cell following administration of a nucleic acid vector encoding the tumor antigen or the tumor antigen itself in the form of a peptide or polypeptide, for example.

As used herein, an “antigen” is a molecule (such as a polypeptide) or a portion thereof that produces an immune response in a host to whom the antigen has been administered. The immune response may include the production of antibodies that bind to at least one epitope of the antigen and/or the generation of a cellular immune response against cells expressing an epitope of the antigen. The response may be an enhancement of a current immune response by, for example, causing increased antibody production, production of antibodies with increased affinity for the antigen, or an increased cellular response. For instance, a suitable immune response may be a humoral response (i.e., antibodies), cellular immune response (i.e., activation of T-helper cells, T-cytotoxic cells, macrophages, and/or natural killer cells), or the elicitation of cytokine, lymphokine and chemokine responses. An antigen that produces an immune response may alternatively be referred to as being immunogenic or as an immunogen. In describing the present invention, a TA may be referred to as an “immunogenic target”.

TA includes both tumor-associated antigens (TAAs) and tumor-specific antigens (TSAs), where a cancerous cell is the source of the antigen. A TAA is an antigen that is expressed on the surface of a tumor cell in higher amounts than is observed on normal cells or an antigen that is expressed on normal cells during fetal development. A TSA is an antigen that is unique to tumor cells and is not expressed on normal cells. TA further includes TAAs or TSAs, antigenic fragments thereof, and modified versions that retain their antigenicity.

TAs are typically classified into five categories according to their expression pattern, function, or genetic origin: cancer-testis (CT) antigens (i.e., MAGE, NY-ESO-1); melanocyte differentiation antigens (i.e., Melan A/MART-1, tyrosinase, gp100); mutational antigens (i.e., MUM-1, p53, CDK-4); overexpressed ‘self’ antigens (i.e., HER-2/neu, p53); and, viral antigens (i.e., HPV, EBV). For the purposes of practicing the present invention, a suitable TA is any TA that induces or enhances an anti-tumor immune response in a host to whom the TA has been administered (i.e., an immunogenic target). Suitable TAs include, for example, gp100 (Cox et al., Science, 264:716-719 (1994)), MART-1/Melan A (Kawakami et al., J. Exp. Med., 180:347-352 (1994)), gp75 (TRP-1) (Wang et al., J. Exp. Med., 186:1131-1140 (1996)), tyrosinase (Wolfel et al., Eur. J. Immunol., 24:759-764 (1994); WO 200175117; WO 200175016; WO 200175007), NY-ESO-1 (WO 98/14464; WO 99/18206), melanoma proteoglycan (Hellstrom et al., J. Immunol., 130:1467-1472 (1983)), MAGE family antigens (i.e., MAGE-1, 2,3,4,6,12, 51; Van der Bruggen et al., Science, 254:1643-1647 (1991); U.S. Pat. Nos. 6,235,525; CN 1319611), BAGE family antigens (Boel et al., Immunity, 2:167-175 (1995)), GAGE family antigens (i.e., GAGE-1,2; Van den Eynde et al., J. Exp. Med., 182:689-698 (1995); U.S. Pat. No. 6,013,765), RAGE family antigens (i.e., RAGE-1; Gaugler et at., Immunogenetics, 44:323-330 (1996); U.S. Pat. No. 5,939,526), N-acetylglucosaminyltransferase-V (Guilloux et at., J. Exp. Med., 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad. Sci. USA, 92:7976-7980 (1995)), cyclin dependent kinase-4 (CDK4) (Wolfel et al., Science, 269:1281-1284 (1995)), p21-ras (Fossum et at., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), p185 HER2/neu (erb-B1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome et al., J. Immunol., 151:1654-1662 (1993)); EBNA gene products of EBV (i.e., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun 2000 September 7;275(3):731-8), HIP-55, TGFβ-1 anti-apoptotic factor (Toomey, et al. Br J Biomed Sci 2001;58(3):177-83), tumor protein D52 (Bryne J. A., et al., Genomics, 35:523-532 (1996)), H1FT, NY-BR-1 (WO 01/47959), NY-BR-62, NY-BR-75, NY-BR-85, NY-BR-87, NY-BR-96 (Scanlan, M. Serologic and Bioinformatic Approaches to the Identification of Human Tumor Antigens, in Cancer Vaccines 2000, Cancer Research Institute, New York, N.Y.), AAC2-1, or AAC2-2, including “wild-type” (i.e., normally encoded by the genome, naturally-occurring), modified, and mutated versions as well as other fragments and derivatives derived therefrom. Any of these TAs may be utilized alone or in combination with one another in a co-immunization protocol.

In one embodiment, the TA is one or more of several MAGE-A1 peptides and variants thereof capable of inducing an immune response in HLA-A2 individuals. The peptides correspond to, or otherwise mimic, epitopes of the MAGE-A1 antigen that, when presented in the context of HLA-A2, induce a cell mediated immune response by interaction with T cells of the immune system, particularly antigen specific cytotoxic CD8+ T cells. This interaction activates the T cells to prevent, eliminate or reduce the occurrence of melanoma in a mammal, including humans.

As used herein, the phrases “MAGE-A1 derived peptide(s)” and “MAGE-A1 peptide(s)” are interchangeable and meant to designate and/or define a chain of amino acids of at least 8 contiguous amino acids, but less than the full-length of the MAGE-A1 protein. As such, it will be appreciated by one of ordinary skill in the art that peptides may also be of a variable length greater than 8 amino acids. In one embodiment of the invention, the MAGE-A1 peptides of the invention comprise 9 amino acids. In preferred embodiments, the TA is a peptide illustrated in SEQ ID NO. 1,2,3 or 4.

In certain cases, it may be beneficial to co-immunize patients with both TA and other antigens, such as angiogenesis-associated antigens (“AA”). An AA is an immunogenic molecule (i.e., peptide, polypeptide) associated with cells involved in the induction and/or continued development of blood vessels. For example, an AA may be expressed on an endothelial cell (“EC”), which is a primary structural component of blood vessels. Where the cancer is cancer, it is preferred that that the AA be found within or near blood vessels that supply a tumor. Immunization of a patient against an AA preferably results in an anti-AA immune response whereby angiogenic processes that occur near or within tumors are prevented and/or inhibited.

Exemplary AAs include, for example, vascular endothelial growth factor (i.e., VEGF; Bernardini, et al. J. Urol., 2001, 166(4): 1275-9; Starnes, et al. J. Thorac. Cardiovasc. Surg., 2001, 122(3): 518-23; Dias, et al. Blood, 2002, 99: 2179-2184), the VEGF receptor (i.e., VEGF-R, flk-1/KDR; Starnes, et al. J. Thorac. Cardiovasc. Surg., 2001, 122(3): 518-23), EPH receptors (i.e., EPHA2; Gerety, et al. 1999, Cell, 4: 403-414), epidermal growth factor receptor (i.e., EGFR; Ciardeillo, et al. Clin. Cancer Res., 2001, 7(10): 2958-70), basic fibroblast growth factor (i.e., bFGF; Davidson, et al. Clin. Exp. Metastasis 2000,18(6): 501-7; Poon, et al. Am J. Surg., 2001, 182(3):298-304), platelet-derived cell growth factor (i.e., PDGF-B), platelet-derived endothelial cell growth factor (PD-ECGF; Hong, et al. J. Mol. Med., 2001, 8(2):141-8), transforming growth factors (i.e., TGF-α; Hong, et al. J. Mol. Med., 2001, 8(2):141-8), endoglin (Balza, et al. Int. J. Cancer, 2001, 94: 579-585), Id proteins (Benezra, R. Trends Cardiovasc. Med., 2001, 11(6):237-41), proteases such as uPA, uPAR, and matrix metalloproteinases (MMP-2, MMP-9; Djonov, et al. J. Pathol., 2001, 195(2):147-55), nitric oxide synthase (Am. J. Ophthalmol., 2001, 132(4):551-6), aminopeptidase (Rouslhati, E. Nature Cancer, 2: 84-90, 2002), thrombospondins (i.e., TSP-1, TSP-2; Alvarez, et al. Gynecol. Oncol., 2001, 82(2):273-8; Seki, et al. Int. J. Oncol., 2001, 19(2):305-10), k-ras (Zhang, et al. Cancer Res., 2001, 61(16):6050-4), Wnt (Zhang, et al. Cancer Res., 2001, 61(16):6050-4), cyclin-dependent kinases (CDKs; Drug Resist. Updat. 2000, 3(2):83-88), microtubules (Timar, et al. 2001. Path. Oncol. Res., 7(2): 85-94), heat shock proteins (i.e., HSP90 (Timar, supra)), heparin-binding factors (i.e., heparinase; Gohji, et al. Int. J. Cancer, 2001, 95(5):295-301), synthases (i.e., ATP synthase, thymidilate synthase), collagen receptors, integrins (i.e., ανβ3, ανβ5, α1β1, α2β1, α5β1), the surface proteolglycan NG2, AAC2-1, or AAC2-2, among others, including “wild-type” (i.e., normally encoded by the genome, naturally-occurring), modified, mutated versions as well as other fragments and derivatives thereof. Any of these targets may be suitable in practicing the present invention, either alone or in combination with one another or with other agents.

In certain embodiments, a nucleic acid molecule encoding the TA is utilized. The nucleic acid molecule may comprise or consist of a nucleotide sequence encoding one or more TAs, or fragments or derivatives thereof, such as that contained in a DNA insert in an ATCC Deposit. The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine, among others.

The nucleic acid sequences described herein represent certain preferred embodiments. Due to degeneracy in the genetic code, variations in the DNA sequence will result in translation of identical peptides. It is thus understood that numerous choices of nucleotides may be made that will lead to a sequence capable of directing production of the peptides or functional analogs thereof of the present invention. As a result, degenerative nucleotide substitutions are included in the scope of the invention. Preferably, the substitution results in expression of a peptide that is recognized by MHC-restricted T cells which specifically recognize cells which have been incubated in the presence of, or “pulsed” with peptides having a sequence selected from any one of SEQ.ID.NO.1, SEQ.ID.NO.2, SEQ.ID.NO.3, SEQ.ID.NO.4, or combinations thereof.

An isolated nucleic acid molecule is one that: (1) is separated from at least about 50 percent of proteins, lipids, carbohydrates, or other materials with which it is naturally found when total nucleic acid is isolated from the source cells; (2) is not be linked to all or a portion of a polynucleotide to which the nucleic acid molecule is linked in nature; (3) is operably linked to a polynucleotide which it is not linked to in nature; and/or, (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. As used herein, the term “naturally occurring” or “native” or “naturally found” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The identity of two or more nucleic acid or polypeptide molecules is determined by comparing the sequences. As known in the art, “identity” means the degree of sequence relatedness between nucleic acid molecules or polypeptides as determined by the match between the units making up the molecules (i.e., nucleotides or amino acid residues). Identity measures the percent of identical matches between the smaller of two or more sequences with gap alignments (if any) addressed by a particular mathematical model or computer program (i.e., an algorithm). Identity between nucleic acid sequences may also be determined by the ability of the related sequence to hybridize to the nucleic acid sequence or isolated nucleic acid molecule. In defining such sequences, the term “highly stringent conditions” and “moderately stringent conditions” refer to procedures that permit hybridization of nucleic acid strands whose sequences are complementary, and to exclude hybridization of significantly mismatched nucleic acids. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. (see, for example, Sambrook, Fritsch & Maniatis, Molecular Cloning: A Laboratory Manual (2nd ed., Cold Spring Harbor Laboratory, 1989); Anderson et al., Nucleic Acid Hybridisation: A Practical Approach Ch. 4 (IRL Press Limited)). The term “moderately stringent conditions” refers to conditions under which a DNA duplex with a greater degree of base pair mismatching than could occur under “highly stringent conditions” is able to form. Exemplary moderately stringent conditions are 0.015 M sodium chloride, 0.0015 M sodium citrate at 50-65° C. or 0.015 M sodium chloride, 0.0015 M sodium citrate, and 20% formamide at 37-50° C. By way of example, moderately stringent conditions of 50° C. in 0.015 M sodium ion will allow about a 21% mismatch. During hybridization, other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH.

In preferred embodiments of the present invention, vectors are used to transfer a nucleic acid sequence encoding a polypeptide to a cell. A vector is any molecule used to transfer a nucleic acid sequence to a host cell. In certain cases, an expression vector is utilized. An expression vector is a nucleic acid molecule that is suitable for transformation of a host cell and contains nucleic acid sequences that direct and/or control the expression of the transferred nucleic acid sequences. Expression includes, but is not limited to, processes such as transcription, translation, and splicing, if introns are present. Expression vectors typically comprise one or more flanking sequences operably linked to a heterologous nucleic acid sequence encoding a polypeptide. Flanking sequences may be homologous (i.e., from the same species and/or strain as the host cell), heterologous (i.e., from a species other than the host cell species or strain), hybrid (i.e., a combination of flanking sequences from more than one source), or synthetic, for example.

A flanking sequence is preferably capable of effecting the replication, transcription and /or translation of the coding sequence and is operably linked to a coding sequence. As used herein, the term operably linked refers to a linkage of polynucleotide elements in a functional relationship. For instance, a promoter or enhancer is operably linked to a coding sequence if it affects the transcription of the coding sequence. However, a flanking sequence need not necessarily be contiguous with the coding sequence, so long as it functions correctly. Thus, for example, intervening untranslated yet transcribed sequences can be present between a promoter sequence and the coding sequence and the promoter sequence may still be considered operably linked to the coding sequence. Similarly, an enhancer sequence may be located upstream or downstream from the coding sequence and affect transcription of the sequence.

In certain embodiments, it is preferred that the flanking sequence is a transcriptional regulatory region that drives high-level gene expression in the target cell. The transcriptional regulatory region may comprise, for example, a promoter, enhancer, silencer, repressor element, or combinations thereof. The transcriptional regulatory region may be either constitutive, tissue-specific, cell-type specific (i.e., the region is drives higher levels of transcription in a one type of tissue or cell as compared to another), or regulatable (i.e., responsive to interaction with a compound such as tetracycline). The source of a transcriptional regulatory region may be any prokaryotic or eukaryotic organism, any vertebrate or invertebrate organism, or any plant, provided that the flanking sequence functions in a cell by causing transcription of a nucleic acid within that cell. A wide variety of transcriptional regulatory regions may be utilized in practicing the present invention.

Suitable transcriptional regulatory regions include the CMV promoter (i.e., the CMV-immediate early promoter); promoters from eukaryotic genes (i.e., the estrogen-inducible chicken ovalbumin gene, the interferon genes, the gluco-corticoid-inducible tyrosine aminotransferase gene, and the thymidine kinase gene); and the major early and late adenovirus gene promoters; the SV40 early promoter region (Bermoist and Chambon, 1981, Nature 290:304-10); the promoter contained in the 3′ long terminal repeat (LTR) of Rous sarcoma virus (RSV) (Yamamoto, et al., 1980, Cell 22:787-97); the herpes simplex virus thymidine kinase (HSV-TK) promoter (Wagner et al., 1981, Proc. Natl. Acad. Sci. U.S.A. 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. U.S.A., 75:3727-31); or the tac promoter (DeBoer et al., 1983, Proc. Natl. Acad. Sci. U.S.A., 80:21-25). Tissue- and/or cell-type specific transcriptional control regions include, for example, the elastase I gene control region which is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region which is active in pancreatic beta cells (Hanahan, 1985, Nature 315:115-22); the immunoglobulin gene control region which is active in lymphoid cells (Grosschedl et al., 1984, Cell 38:647-58; Adames et al., 1985, Nature 318:533-38; Alexander et al., 1987, Mol. Cell. Biol, 7:1436-44); the mouse mammary tumor virus control region in testicular, breast, lymphoid and mast cells (Leder et al., 1986, Cell 45:485-95); the albumin gene control region in liver (Pinkert et al., 1987, Genes and Devel. 1:268-76); the alpha-feto-protein gene control region in liver (Krumlauf et al., 1985, Mol. Cell. Biol., 5:1639-48; Hammer et al., 1987, Science 235:53-58); the alpha 1-antitrypsin gene control region in liver (Kelsey et al., 1987, Genes and Devel. 1:161-71); the beta-globin gene control region in myeloid cells (Mogram et al., 1985, Nature 315:338-40; Kollias et al., 1986, Cell 46:89-94); the myelin basic protein gene control region in oligodendrocyte cells in the brain (Readhead et al., 1987, Cell 48:703-12); the myosin light chain-2 gene control region in skeletal muscle (Sani, 1985, Nature 314:283-86); the gonadotropic releasing hormone gene control region in the hypothalamus (Mason et al., 1986, Science 234:1372-78), and the tyrosinase promoter in melanoma cells (Hart, I. Semin Oncol 1996 February;23(1):154-8; Siders, et al. Cancer Gene Ther 1998 September-October;5(5):281-91), among others. Other suitable promoters are known in the art.

As described above, enhancers may also be suitable flanking sequences. Enhancers are cis-acting elements of DNA, usually about 10-300 bp in length, that act on the promoter to increase transcription. Enhancers are typically orientation- and position-independent, having been identified both 5′ and 3′ to controlled coding sequences. Several enhancer sequences available from mammalian genes are known (i.e., globin, elastase, albumin, alpha-feto-protein and insulin). Similarly, the SV40 enhancer, the cytomegalovirus early promoter enhancer, the polyoma enhancer, and adenovirus enhancers are useful with eukaryotic promoter sequences. While an enhancer may be spliced into the vector at a position 5′ or 3′ to nucleic acid coding sequence, it is typically located at a site 5′ from the promoter. Other suitable enhancers are known in the art, and would be applicable to the present invention.

While preparing reagents of the present invention, cells may need to be transfected or transformed. Transfection refers to the uptake of foreign or exogenous DNA by a cell, and a cell has been transfected when the exogenous DNA has been introduced inside the cell membrane. A number of transfection techniques are well known in the art (i.e., Graham et al., 1973, Virology 52:456; Sambrook et al., Molecular Cloning, A Laboratory Manual (Cold Spring Harbor Laboratories, 1989); Davis et al., Basic Methods in Molecular Biology (Elsevier, 1986); and Chu et al., 1981, Gene 13:197). Such techniques can be used to introduce one or more exogenous DNA moieties into suitable host cells.

In certain embodiments, it is preferred that transfection of a cell results in transformation of that cell. A cell is transformed when there is a change in a characteristic of the cell, being transformed when it has been modified to contain a new nucleic acid. Following transfection, the transfected nucleic acid may recombine with that of the cell by physically integrating into a chromosome of the cell, may be maintained transiently as an episomal element without being replicated, or may replicate independently as a plasmid. A cell is stably transformed when the nucleic acid is replicated with the division of the cell.

The present invention further provides isolated TAs in polypeptide form. A polypeptide is considered isolated where it: (1) has been separated from at least about 50 percent of polynucleotides, lipids, carbohydrates, or other materials with which it is naturally found when isolated from the source cell; (2) is not linked (by covalent or noncovalent interaction) to all or a portion of a polypeptide to which the “isolated polypeptide” is linked in nature; (3) is operably linked (by covalent or noncovalent interaction) to a polypeptide with which it is not linked in nature; or, (4) does not occur in nature. Preferably, the isolated polypeptide is substantially free from any other contaminating polypeptides or other contaminants that are found in its natural environment that would interfere with its therapeutic, diagnostic, prophylactic or research use.

TA polypeptides may be mature polypeptides, as defined herein, and may or may not have an amino terminal methionine residue, depending on the method by which they are prepared. Further contemplated are related polypeptides such as, for example, fragments, variants (i.e., allelic, splice), orthologs, homologues, and derivatives, for example, that possess at least one characteristic or activity (i.e., activity, antigenicity) of the TA. Also related are peptides, which refers to a series of contiguous amino acid residues having a sequence corresponding to at least a portion of the polypeptide from which its sequence is derived. In preferred embodiments, the peptide comprises about 5-10 amino acids, 10-15 amino acids, 15-20 amino acids, 20-30 amino acids, or 30-50 amino acids. In a more preferred embodiment, a peptide comprises 9-12 amino acids, suitable for presentation upon Class I MHC molecules, for example.

A fragment of a nucleic acid or polypeptide comprises a truncation of the sequence (i.e., nucleic acid or polypeptide) at the amino terminus (with or without a leader sequence) and/or the carboxy terminus. Fragments may also include variants (i.e., allelic, splice), orthologs, homologues, and other variants having one or more amino acid additions or substitutions or internal deletions as compared to the parental sequence. In preferred embodiments, truncations and/or deletions comprise about 10 amino acids, 20 amino acids, 30 amino acids, 40 amino acids, 50 amino acids, or more. The polypeptide fragments so produced will comprise about 10 amino acids, 25 amino acids, 30 amino acids, 40 amino acids, 50 amino acids, 60 amino acids, 70 amino acids, or more. Such polypeptide fragments may optionally comprise an amino terminal methionine residue. It will be appreciated that such fragments can be used, for example, to generate antibodies or cellular immune responses to TA polypeptides.

A variant is a sequence having one or more sequence substitutions, deletions, and/or additions as compared to the subject sequence. Variants may be naturally occurring or artificially constructed. Such variants may be prepared from the corresponding nucleic acid molecules. In preferred embodiments, the variants have from 1 to 3, or from 1 to 5, or from 1 to 10, or from 1 to 15, or from 1 to 20, or from 1 to 25, or from 1 to 30, or from 1 to 40, or from 1 to 50, or more than 50 amino acid substitutions, insertions, additions and/or deletions.

An allelic variant is one of several possible naturally-occurring alternate forms of a gene occupying a given locus on a chromosome of an organism or a population of organisms. A splice variant is a polypeptide generated from one of several RNA transcript resulting from splicing of a primary transcript. An ortholog is a similar nucleic acid or polypeptide sequence from another species. For example, the mouse and human versions of a TA polypeptide may be considered orthologs of each other. A derivative of a sequence is one that is derived from a parental sequence those sequences having substitutions, additions, deletions, or chemically modified variants. Variants may also include fusion proteins, which refers to the fusion of one or more first sequences (such as a peptide) at the amino or carboxy terminus of at least one other sequence (such as a heterologous peptide).

“Similarity” is a concept related to identity, except that similarity refers to a measure of relatedness which includes both identical matches and conservative substitution matches. If two polypeptide sequences have, for example, 10/20 identical amino acids, and the remainder are all non-conservative substitutions, then the percent identity and similarity would both be 50%. If in the same example, there are five more positions where there are conservative substitutions, then the percent identity remains 50%, but the percent similarity would be 75% (15/20). Therefore, in cases where there are conservative substitutions, the percent similarity between two polypeptides will be higher than the percent identity between those two polypeptides.

Substitutions may be conservative, or non-conservative, or any combination thereof. Conservative amino acid modifications to the sequence of a polypeptide (and the corresponding modifications to the encoding nucleotides) may produce polypeptides having functional and chemical characteristics similar to those of a parental polypeptide. For example, a “conservative amino acid substitution” may involve a substitution of a native amino acid residue with a non-native residue such that there is little or no effect on the size, polarity, charge, hydrophobicity, or hydrophilicity of the amino acid residue at that position and, in particlar, does not result in decreased immunogenicity. Suitable conservative amino acid substitutions are shown in Table I.

TABLE I

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of polypeptide using well-known techniques. For identifying suitable areas of the molecule that may be changed without destroying biological activity (i.e., MHC binding, immunogenicity), one skilled in the art may target areas not believed to be important for that activity. For example, when similar polypeptides with similar activities from the same species or from other species are known, one skilled in the art may compare the amino acid sequence of a polypeptide to such similar polypeptides. By performing such analyses, one can identify residues and portions of the molecules that are conserved among similar polypeptides. It will be appreciated that changes in areas of the molecule that are not conserved relative to such similar polypeptides would be less likely to adversely affect the biological activity and/or structure of a polypeptide.

Similarly, the residues required for binding to MHC are known, and may be modified to improve binding. For instance, the MAGE-1 peptides of the present invention may be derived from the peptides of SEQ.ID.NO.1, SEQ.ID.NO.2, SEQ.ID.NO.3 or SEQ.ID.NO.4, and contain non-conservative amino acid changes at one or more positions to the extent that such peptides are capable of presentation by HLA-A2 determinants and elicit an anti-MAGE-1 response. However, modifications resulting in decreased binding to MHC will not be appropriate in most situations. One skilled in the art would also know that, even in relatively conserved regions, one may substitute chemically similar amino acids for the naturally occurring residues while retaining activity. Therefore, even areas that may be important for biological activity or for structure may be subject to conservative amino acid substitutions without destroying the biological activity or without adversely affecting the polypeptide structure.

Other preferred polypeptide variants include glycosylation variants wherein the number and/or type of glycosylation sites have been altered compared to the subject amino acid sequence. In one embodiment, polypeptide variants comprise a greater or a lesser number of N-linked glycosylation sites than the subject amino acid sequence. An N-linked glycosylation site is characterized by the sequence Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. To affect O-linked glycosylation of a polypeptide, one would modify serine and/or threonine residues.

Additional preferred variants include cysteine variants, wherein one or more cysteine residues are deleted or substituted with another amino acid (e.g., serine) as compared to the subject amino acid sequence set. Cysteine variants are useful when polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines.

In other embodiments, the isolated polypeptides of the current invention include fusion polypeptide segments that assist in purification of the polypeptides. Fusions can be made either at the amino terminus or at the carboxy terminus of the subject polypeptide variant thereof. Fusions may be direct with no linker or adapter molecule or may be through a linker or adapter molecule. A linker or adapter molecule may be one or more amino acid residues, typically from about 20 to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derivatized according to the methods described herein. Suitable fusion segments include, among others, metal binding domains (e.g., a poly-histidine segment), immunoglobulin binding domains (i.e., Protein A, Protein G, T cell, B cell, Fc receptor, or complement protein antibody-binding domains), sugar binding domains (e.g., a maltose binding domain), and/or a “tag” domain (i.e., at least a portion of α-galactosidase, a strep tag peptide, a T7 tag peptide, a FLAG peptide, or other domains that can be purified using compounds that bind to the domain, such as monoclonal antibodies). This tag is typically fused to the polypeptide upon expression of the polypeptide, and can serve as a means for affinity purification of the sequence of interest polypeptide from the host cell. Affinity purification can be accomplished, for example, by column chromatography using antibodies against the tag as an affinity matrix. Optionally, the tag can subsequently be removed from the purified sequence of interest polypeptide by various means such as using certain peptidases for cleavage. As described below, fusions may also be made between a TA and a co-stimulatory components such as the chemokines CXC10 (IP-10), CCL7 (MCP-3), or CCL5 (RANTES), for example.

A fusion motif may enhance transport of a TA to an MHC processing compartment, such as the endoplasmic reticulum. These sequences, referred to as tranduction or transcytosis sequences, include sequences derived from HIV tat (see Kim et al. 1997 J. Immunol. 159:1666), Drosophila antennapedia (see Schutze-Redelmeier et al. 1996 J. Immunol. 157:650), or human period-1 protein (hPER1; in particular, SRRHHCRSKAKRSRHH).

In addition, the polypeptide or variant thereof may be fused to a homologous polypeptide to form a homodimer or to a heterologous polypeptide to form a heterodimer. Heterologous peptides and polypeptides include, but are not limited to: an epitope to allow for the detection and/or isolation of a fusion polypeptide; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the polypeptide or variant thereof.

In a further aspect, lipopeptide derivatives may be utilized. Lipopeptides enhance the induction of CTL responses against antigens in vivo (see for e.g. Deres et al., Nature 342, 561-564 (1989)) and constitute potent adjuvants in parenteral and mucosal immunization (Baier et al., Immunobiology 201, 391-405 (2000)). For example, the lipopeptides may comprise a MAGE-A1 HLA-A2 peptide and one or more chains derived from fatty acids and/or steroid groups, and also include synthetic lipopeptides. The lipopeptides may be prepared using techniques known in the art (Loing et al., J. Immunol. 164(2), 900-907 (2000)). In particular, the fatty acids and/or steroid groups may be coupled on the alpha-NH₂ or epsilon-NH₂ functional groups of the amino acid residues of the MAGE-A1 HLA-A2 peptide.

The present invention also contemplates nonpeptide analogs of the peptides of the invention, e.g. peptide mimetics, that provide a stabilized structure or lessened biodegradation. Peptide mimetic analogs can be prepared based on a selected MAGE-A1 HLA-A2 peptide having a sequence selected from SEQ ID NO. 1, SEQ ID NO. 2, SEQ ID. NO. 3 and SEQ ID NO. 4 by replacement of one or more residues by non-peptide moieties. Preferably, the nonpeptide moieties permit the peptide to retain its natural conformation, or stabilize a preferred, e.g. bioactive confirmation. Such peptides can be tested in molecular or cell-based binding assays to assess the effect of the substitution(s) on conformation and/or activity. The preparation of nonpeptide mimetic analogs from the peptides of the invention can be done, for example, as taught in Nachman et al., Regul Pept.57:359-370 (1995).

In certain embodiments, it may be advantageous to combine a nucleic acid sequence encoding a TA, polypeptide, or derivative thereof with one or more co-stimulatory component(s) such as cell surface proteins, cytokines or chemokines in a composition of the present invention. The co-stimulatory component may be included in the composition as a polypeptide or as a nucleic acid encoding the polypeptide, for example. Suitable co-stimulatory molecules include, for instance, polypeptides that bind members of the CD28 family (i.e., CD28, ICOS; Hutloff, et al. Nature 1999, 397: 263-265; Peach, et al. J Exp Med 1994, 180: 2049-2058) such as the CD28 binding polypeptides B7.1 (CD80; Schwartz, 1992; Chen et al, 1992; Ellis, et al. J. Immunol., 156(8): 2700-9) and B7.2 (CD86; Ellis, et al. J. Immunol., 156(8): 2700-9); polypeptides which bind members of the integrin family (i.e., LFA-1 (CD 11a/CD18); Sedwick, et al. J Immunol 1999, 162: 1367-1375; Wülfing, et al. Science 1998, 282: 2266-2269; Lub, et al. Immunol Today 1995, 16: 479-483) including members of the ICAM family (i.e., ICAM-1, -2 or -3); polypeptides which bind CD2 family members (i.e., CD2, signalling lymphocyte activation molecule (CDw150 or “SLAM”; Aversa, et al. J Immunol 1997, 158: 4036-4044)) such as CD58 (LFA-3; CD2 ligand; Davis, et al. Immunol Today 1996, 17: 177-187) or SLAM ligands (Sayos, et al. Nature 1998, 395: 462-469); polypeptides which bind heat stable antigen (HSA or CD24; Zhou, et al. Eur J Immunol 1997, 27: 2524-2528); polypeptides which bind to members of the TNF receptor (TNFR) family (i.e., 4-1BB (CD137; Vinay, et al. Semin Immunol 1998, 10: 481-489), OX40 (CD134; Weinberg, et al. Semin Immunol 1998, 10: 471-480; Higgins, et al. J Immunol 1999, 162: 486-493), and CD27 (Lens, et al. Semin Immunol 1998, 10: 491-499)) such as 4-1BBL (4-1BB ligand; Vinay, et al. Semin Immunol 1998, 10: 481-48; DeBenedette, et al. J Immunol 1997, 158: 551-559), TNFR associated factor-1 (TRAF-1; 4-1BB ligand; Saoulli, et al. J Exp Med 1998, 187: 1849-1862, Arch, et al. Mol Cell Biol 1998, 18: 558-565), TRAF-2 (4-1BB and OX40 ligand; Saoulli, et al. J Exp Med 1998, 187: 1849-1862; Oshima, et al. Int Immunol 1998, 10: 517-526, Kawamata, et al. J Biol Chem 1998, 273: 5808-5814), TRAF-3 (4-1BB and OX40 ligand; Arch, et al. Mol Cell Biol 1998, 18: 558-565; Jang, et al. Biochem Biophys Res Commun 1998, 242: 613-620; Kawamata S, et al. J Biol Chem 1998, 273: 5808-5814), OX40L (OX40 ligand; Gramaglia, et al. J Immunol 1998, 161: 6510-6517), TRAF-5 (OX40 ligand; Arch, et al. Mol Cell Biol 1998, 18: 558-565; Kawamata, et al. J Biol Chem 1998, 273: 5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161: 4563-4571; Sine, et al. Hum. Gene Ther., 2001, 12: 1091-1102) may also be suitable.

One or more cytokines may also be suitable co-stimulatory components or “adjuvants”, either as polypeptides or being encoded by nucleic acids contained within the compositions of the present invention (Parmiani, et al. Immunol Lett 2000 September 15; 74(1): 41-4; Berzofsky, et al. Nature Immunol. 1: 209-219). Suitable cytokines include, for example, interleukin-2 (IL-2) (Rosenberg, et al. Nature Med. 4: 321-327 (1998)), IL-4, IL-7, IL-12 (reviewed by Pardoll, 1992; Harries, et al. J. Gene Med. 2000 July-August;2(4):243-9; Rao, et al. J. Immunol. 156: 3357- 3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), tumor necrosis factor-alpha (TNF-α), or interferons such as IFN-α or INF-γ. Other cytokines may also be suitable for practicing the present invention, as is known in the art.

Chemokines may also be utilized. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258). The chemokines CCL3 (MIP-1α) and CCL5 (RANTES) (Boyer, et al. Vaccine, 1999, 17 (Supp. 2): S53-S64) may also be of use in practicing the present invention. Other suitable chemokines are known in the art.

It is also known in the art that suppressive or negative regulatory immune mechanisms may be blocked, resulting in enhanced immune responses. For instance, treatment with anti-CTLA-4 (Shrikant, et al. Immunity, 1996, 14: 145-155; Sutmuller, et al. J. Exp. Med., 2001, 194: 823-832), anti-CD25 (Sutmuller, supra), anti-CD4 (Matsui, et al. J. Immunol., 1999, 163: 184-193), the fusion protein IL13Ra2-Fc (Terabe, et al. Nature Immunol., 2000, 1: 515-520), and combinations thereof (i.e., anti-CTLA-4 and anti-CD25, Sutmuller, supra) have been shown to upregulate anti-tumor immune responses and would be suitable in practicing the present invention.

Any of these components may be used alone or in combination with other agents. For instance, it has been shown that a combination of CD80, ICAM-1 and LFA-3 (“TRICOM”) may potentiate anti-cancer immune responses (Hodge, et al. Cancer Res. 59: 5800-5807 (1999). Other effective combinations include, for example, IL-12+GM-CSF (Ahlers, et al. J. Immunol., 158: 3947-3958 (1997); Iwasaki, et al. J. Immunol. 158: 4591-4601 (1997)), IL-12+GM-CSF+TNF-α (Ahlers, et al. Int. Immunol. 13: 897-908 (2001)), CD80+IL-12 (Fruend, et al. Int. J. Cancer, 85: 508-517 (2000); Rao, et al. supra), and CD86+GM-CSF+IL-12 (Iwasaki, supra). One of skill in the art would be aware of additional combinations useful in carrying out the present invention.In addition, the skilled artisan would be aware of additional reagents or methods that may be used to modulate such mechanisms. These reagents and methods, as well as others known by those of skill in the art, may be utilized in practicing the present invention.

The TAs of the present invention and analogs thereof may also be used in the preparation of passive immunotherapy agents such as immune cells or antibodies with antitumor reactivity. The nucleic acid sequences encoding the MAGE-A1 peptides sequences, for example, can be expressed alone or in combination with other components in genetically modified immune cells. Alternatively, antigen presenting cells may be pulsed with the peptides to provide stimulator cells for the activation of tumor specific cytotoxic T cells. In this embodiment, autologous cytotoxic lymphocytes or tumor infiltrating lymphocytes are obtained from a patient with cancer. The lymphocytes are grown in culture and antigen specific lymphocytes are expanded by culturing in the presence of antigen presenting cells which present the peptides in the context of HLA-A molecules. The antigen specific lymphocytes are then infused back into the patient in an amount effective to reduce or eliminate the tumors in the patient.

Additional strategies for improving the efficiency of nucleic acid-based immunization may also be used including, for example, the use of self-replicating viral replicons (Caley, et al. 1999. Vaccine, 17: 3124-2135; Dubensky, et al. 2000. Mol. Med. 6: 723-732; Leitner, et al. 2000. Cancer Res. 60: 51-55), codon optimization (Liu, et al. 2000. Mol. Ther., 1: 497-500; Dubensky, supra; Huang, et al. 2001. J. Virol. 75: 4947-4951), in vivo electroporation (Widera, et al. 2000. J. Immunol. 164: 4635-3640), incorporation of CpG stimulatory motifs (Gurunathan, et al. Ann. Rev. Immunol., 2000, 18: 927-974; Leitner, supra), sequences for targeting of the endocytic or ubiquitin-processing pathways (Thomson, et al. 1998. J. Virol. 72: 2246-2252; Velders, et al. 2001. J. Immunol. 166: 5366-5373), Marek's disease virus type 1 VP22 sequences (J. Virol. 76(6):2676-82, 2002), prime-boost regimens (Gurunathan, supra; Sullivan, et al. 2000. Nature, 408: 605-609; Hanke, et al. 1998. Vaccine, 16: 439-445; Amara, et al. 2001. Science, 292: 69-74), and the use of mucosal delivery vectors such as Salmonella (Darji, et al. 1997. Cell, 91: 765-775; Woo, et al. 2001. Vaccine, 19: 2945-2954). Other methods are known in the art, some of which are described below.

In certain embodiments, the TAs, such as the MAGE-A1 peptides, may be utilized to treat melanoma. The term “melanoma” includes, but is not limited to, melanomas, metastatic melanomas, melanomas derived from either melanocytes or melanocyte related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAM-M) syndrome.

Chemotherapeutic agents, radiation, anti-angiogenic compounds, or other agents for treating cancer may also be in combination with immunization strategies (Sebti, et al. Oncogene 2000 December 27;19(56):6566-73). Suitable treatments for melanoma include, for example, surgery, adjuvant radiation therapy (Ang et al. Regional radiotherapy as adjuvant treatment for head and neck malignancy melanoma. Arch Otolaryngol Head Neck Surg. 1990;116:169-172), adjuvant interferon alfa-2b (Kirkwood, et al. Interferon alfa-2 b adjuvant therapy of high-risk resected cutaneous melanoma: the Eastern Cooperative Oncology Group Trial 1684. J Clin Oncol. 1996; 14:7-17), corticosteroids, systemic therapy (Amer, et al. Malignant melanoma and central nervous system metastases: incidence, diagnosis, treatment and survival. Cancer, 1978; 42: 660-668), dacarbazine, combination chemotherapies (Del Prete, et al. Combination chemotherapy with cisplatin, carmustine, dacarbazine, and tamoxifen in metastatic melanoma. Cancer Treat Rep. 1984;68:1403-1405; Seigler HF, Lucas VS Jr, Pickett N.J., et al. DTIC, CCNU, bleomycin and vincristine (BOLD) in metastatic melanoma. Cancer. 1980;46:2346-2348). Other suitable treatments are known in the art.

Many anti-angiogenic agents are known in the art and may also be suitable for co-administration with the immunotherapeutic components (see, for example, Timar, et al. 2001. Pathology Oncol. Res., 7(2): 85-94). Such agents include, for example, physiological agents such as growth factors (i.e., ANG-2, NK1,2,4 (HGF), transforming growth factor beta (TGF-β)), cytokines (i.e., interferons such as IFN-α, -β, -γ, platelet factor 4 (PF-4), PR-39), proteases (i.e., cleaved AT-III, collagen XVIII fragment (Endostatin)), HmwKallikrein-d5 plasmin fragment (Angiostatin), prothrombin-F1-2, TSP-1), protease inhibitors (i.e., tissue inhibitor of metalloproteases such as TIMP-1, -2, or -3; maspin; plasminogen activator-inhibitors such as PAI-1; pigment epithelium derived factor (PEDF)), Tumstatin (available through ILEX, Inc.), antibody products (i.e., the collagen-binding antibodies HUIV26, HUI77, XL313; anti-VEGF; anti-integrin (i.e., Vitaxin, (Lxsys))), and glycosidases (i.e., heparinase-I, -III). “Chemical” or modified physiological agents known or believed to have anti-angiogenic potential include, for example, vinblastine, taxol, ketoconazole, thalidomide, dolestatin, combrestatin A, rapamycin (Guba, et al. 2002, Nature Med., 8: 128-135), CEP-7055 (available from Cephalon, Inc.), flavone acetic acid, Bay 12-9566 (Bayer Corp.), AG3340 (Agouron, Inc.), CGS 27023A (Novartis), tetracylcine derivatives (i.e., COL-3 (Collagenix, Inc.)), Neovastat (Aeterna), BMS-275291 (Bristol-Myers Squibb), low dose 5-FU, low dose methotrexate (MTX), irsofladine, radicicol, cyclosporine, captopril, celecoxib, D45152-sulphated polysaccharide, cationic protein (Protamine), cationic peptide-VEGF, Suramin (polysulphonated napthyl urea), compounds that interfere with the function or production of VEGF (i.e., SU5416 or SU6668 (Sugen), PTK787/ZK22584 (Novartis)), Distamycin A, Angiozyme (ribozyme), isoflavinoids, staurosporine derivatives, genistein, EMD121974 (Merck KcgaA), tyrphostins, isoquinolones, retinoic acid, carboxyamidotriazole, TNP-470, octreotide, 2-methoxyestradiol, aminosterols (i.e., squalamine), glutathione analogues (i.e., N-acteyl-L-cysteine), combretastatin A-4 (Oxigene), Eph receptor blocking agents (Nature, 414:933-938, 2001), Rh-Angiostatin, Rh-Endostatin (WO 01/93897), cyclic-RGD peptide, accutin-disintegrin, benzodiazepenes, humanized anti-avb3 Ab, Rh-PAI-2, amiloride, p-amidobenzamidine, anti-uPA ab, anti-uPAR Ab, L-phanylalanin-N-methylamides (i.e., Batimistat, Marimastat), AG3340, and minocycline. Many other suitable agents are known in the art and would suffice in practicing the present invention.

The present invention may also be utilized in combination with “non-traditional” methods of treating cancer. For example, it has recently been demonstrated that administration of certain anaerobic bacteria may assist in slowing tumor growth. In one study, Clostridium novyi was modified to eliminate a toxin gene carried on a phage episome and administered to mice with colorectal tumors (Dang, et al. P.N.A.S. USA, 98(26): 15155-15160, 2001). In combination with chemotherapy, the treatment was shown to cause tumor necrosis in the animals. The reagents and methodologies described in this application may be combined with such treatment methodologies.

Nucleic acids encoding TAs may be administered to patients by any of several available techniques. Various viral vectors that have been successfully utilized for introducing a nucleic acid to a host include retrovirus, adenovirus, adeno-associated virus (AAV), herpes virus, and poxvirus, among others. It is understood in the art that many such viral vectors are available in the art. The vectors of the present invention may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

Preferred retroviral vectors are derivatives of lentivirus as well as derivatives of murine or avian retroviruses. Examples of suitable retroviral vectors include, for example, Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of retroviral vectors can incorporate multiple exogenous nucleic acid sequences. As recombinant retroviruses are defective, they require assistance in order to produce infectious vector particles. This assistance can be provided by, for example, helper cell lines encoding retrovirus structural genes. Suitable helper cell lines include Ψ2, PA317 and PA12, among others. The vector virions produced using such cell lines may then be used to infect a tissue cell line, such as NIH 3T3 cells, to produce large quantities of chimeric retroviral virions. Retroviral vectors may be administered by traditional methods (i.e., injection) or by implantation of a “producer cell line” in proximity to the target cell population (Culver, K., et al., 1994, Hum. Gene Ther., 5 (3): 343-79; Culver, K., et al., Cold Spring Harb. Symp. Quant. Biol., 59: 685-90); Oldfield, E., 1993, Hum. Gene Ther., 4 (1): 39-69). The producer cell line is engineered to produce a viral vector and releases viral particles in the vicinity of the target cell. A portion of the released viral particles contact the target cells and infect those cells, thus delivering a nucleic acid of the present invention to the target cell. Following infection of the target cell, expression of the nucleic acid of the vector occurs.

Adenoviral vectors have proven especially useful for gene transfer into eukaryotic cells (Rosenfeld, M., et al., 1991, Science, 252 (5004): 431-4; Crystal, R., et al., 1994, Nat. Genet., 8 (1): 42-51), the study eukaryotic gene expression (Levrero, M., et al., 1991, Gene, 101 (2): 195-202), vaccine development (Graham, F. and Prevec, L., 1992, Biotechnology, 20: 363-90), and in animal models (Stratford-Perricaudet, L., et al., 1992, Bone Marrow Transplant., 9 (Suppl. 1): 151-2 ; Rich, D., et al., 1993, Hum. Gene Ther., 4 (4): 461-76). Experimental routes for administrating recombinant Ad to different tissues in vivo have included intratracheal instillation (Rosenfeld, M., et al., 1992, Cell, 68 (1): 143-55) injection into muscle (Quantin, B., et al., 1992, Proc. Natl. Acad. Sci. U.S.A., 89 (7): 2581-4), peripheral intravenous injection (Herz, J., and Gerard, R., 1993, Proc. Natl. Acad. Sci. U.S.A., 90 (7): 2812-6) and stereotactic inoculation to brain (Le Gal La Salle, G., et al., 1993, Science, 259 (5097): 988-90), among others.

Adeno-associated virus (AAV) demonstrates high-level infectivity, broad host range and specificity in integrating into the host cell genome (Hermonat, P., et al., 1984, Proc. Natl. Acad. Sci. U.S.A., 81 (20): 6466-70). And Herpes Simplex Virus type-1 (HSV-1) is yet another attractive vector system, especially for use in the nervous system because of its neurotropic property (Geller, A., et al., 1991, Trends Neurosci., 14 (10): 428-32; Glorioso, et al., 1995, Mol. Biotechnol., 4 (1): 87-99; Glorioso, et al., 1995, Annu. Rev. Microbiol., 49: 675-710).

Poxvirus is another useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). Poxviruses shown to be useful include vaccinia, NYVAC, avipox, fowlpox, canarypox, ALVAC, and ALVAC(2), among others.

NYVAC (vP866) was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R); hemorrhagic region (u; B13R+B14R); A type inclusion body region (ATI; A26L); hemagglutinin gene (HA; A56R); host range gene region (C7L-K1L); and, large subunit, ribonucleotide reductase (I4L). NYVAC is a genetically engineered vaccinia virus strain that was generated by the specific deletion of eighteen open reading frames encoding gene products associated with virulence and host range. NYVAC has been show to be useful for expressing TAs (see, for example, U.S. Pat. No. 6,265,189). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively.

ALVAC-based recombinant viruses (i.e., ALVAC-1 and ALVAC-2) are also suitable for use in practicing the present invention (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

Another useful poxvirus vector is TROVAC. TROVAC refers to an attenuated fowlpox that was a plaque-cloned isolate derived from the FP-1 vaccine strain of fowlpoxvirus which is licensed for vaccination of 1 day old chicks. TROVAC was likewise deposited under the terms of the Budapest Treaty with the ATCC, accession number 2553.

“Non-viral” plasmid vectors may also be suitable in practicing the present invention. Preferred plasmid vectors are compatible with bacterial, insect, and/or mammalian host cells. Such vectors include, for example, PCR-II, pCR3, and pcDNA3.1 (Invitrogen, San Diego, Calif.), pBSII (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (PCT pub. No. WO 90/14363) and pFastBacDual (Gibco-BRL, Grand Island, N.Y.) as well as Bluescript® plasmid derivatives (a high copy number COLE1-based phagemid, Stratagene Cloning Systems, La Jolla, Calif.), PCR cloning plasmids designed for cloning Taq-amplified PCR products (e.g., TOPO™ TA cloning® kit, PCR2.1® plasmid derivatives, Invitrogen, Carlsbad, Calif.). Bacterial vectors may also be used with the current invention. These vectors include, for example, Shigella, Salmonella, Vibrio cholerae, Lactobacillus, Bacille calmette guérin (BCG), and Streptococcus (see for example, WO 88/6626; WO 90/0594; WO 91/13157; WO 92/1796; and WO 92/21376). Many other non-viral plasmid expression vectors and systems are known in the art and could be used with the current invention.

Suitable nucleic acid delivery techniques include DNA-ligand complexes, adenovirus-ligand-DNA complexes, direct injection of DNA, CaPO₄ precipitation, gene gun techniques, electroporation, and colloidal dispersion systems, among others. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

A TA may also be administered in combination with one or more adjuvants to boost the immune response. Exemplary adjuvants are shown in Table II below:

TABLE II
Types of Immunologic Adjuvants

| Type of | | | |
|---|---|---|---|
| Adjuvant | | General Examples | Specific Examples/References |
| 1 | Gel-type | Aluminum hydroxide/ | (Aggerbeck and Heron, 1995) |
| | | phosphate (“alum | |
| | | adjuvants”) | |
| | | Calcium phosphate | (Relyveld, 1986) |
| 2 | Microbial | Muramyl dipeptide | (Chedid et al., 1986) |
| | | (MDP) | |
| | | Bacterial exotoxins | Cholera toxin (CT), E. coli |
| | | | labile toxin (LT)(Freytag and |
| | | | Clements, 1999) |
| | | Endotoxin-based | Monophosphoryl lipid A |
| | | adjuvants | (MPL) (Ulrich and Myers, |
| | | | 1995) |
| | | Other bacterial | CpG oligonucleotides (Corral |
| | | | and Petray, 2000), BCG |
| | | | sequences (Krieg, et al. |
| | | | Nature, 374: 576), tetanus |
| | | | toxoid (Rice, et al. J. |
| | | | Immunol., 2001, 167: |
| | | | 1558-1565) |
| 3 | Particulate | Biodegradable | (Gupta et al., 1998) |
| | | Polymer microspheres | |
| | | Immunostimulatory | (Morein and Bengtsson, 1999) |
| | | complexes (ISCOMs) | |
| | | Liposomes | (Wassef et al., 1994) |
| 4 | Oil-emul- | Freund's incomplete | (Jensen et al., 1998) |
| | sion and | adjuvant | |
| | surfactant- | Microfluidized emulsions | MF59 (Ott et al., 1995) |
| | based | | SAF (Allison and Byars, |
| | adjuvants | | 1992) (Allison, 1999) |
| | | Saponins | QS-21 (Kensil, 1996) |
| 5 | Synthetic | Muramyl peptide | Murabutide (Lederer, 1986) |
| | | derivatives | Threony-MDP (Allison, 1997) |
| | | Nonionic block | L121 (Allison, 1999) |
| | | copolymers | |
| | | Polyphosphazene (PCPP) | (Payne et al., 1995) |
| | | Synthetic polynucleotides | Poly A:U, Poly I:C (Johnson, |
| | | | 1994) |

The TAs of the present invention may also be used to generate antibodies for various uses (i.e., screening assays, diagnostic assays, passive immunotherapy). Other uses would be apparent to one of skill in the art. The term “antibody” includes various antibody-related reagents including, for example, antibody fragments (i.e., Fab, Fab₂, single chain antibodies such as Fᵥ), humanized antibodies, chimeric antibodies, or human antibodies.

Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boemer et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). The antibodies or derivatives therefrom may also be conjugated to therapeutic moieties such as cytotoxic drugs or toxins, or active fragments thereof such as diptheria A chain, exotoxin A chain, ricin A chain, abrin A chain, curcin, crotin, phenomycin, enomycin, among others. Cytotoxic agents may also include radiochemicals. Antibodies and their derivatives may be incorporated into compositions of the invention for use in vitro or in vivo.

Nucleic acids, proteins, or derivatives thereof representing a TA may be used in assays to determine the presence of a disease state in a patient, to predict prognosis, or to determine the effectiveness of a chemotherapeutic or other treatment regimen. Expression profiles, performed as is known in the art, may be used to determine the relative level of expression of the TA. The level of expression may then be correlated with base levels to determine whether a particular disease is present within the patient, the patient's prognosis, or whether a particular treatment regimen is effective. For example, if the patient is being treated with a particular chemotherapeutic regimen, an decreased level of expression of a TA in the patient's tissues (i.e., in peripheral blood) may indicate the regimen is decreasing the cancer load in that host. Similarly, if the level of expresssion is increasing, another therapeutic modality may need to be utilized. In one embodiment, nucleic acid probes corresponding to a nucleic acid encoding a TA may be attached to a biochip, as is known in the art, for the detection and quantification of expression in the host.

It is also possible to use nucleic acids, proteins, derivatives therefrom, or antibodies thereto as reagents in drug screening assays. The reagents may be used to ascertain the effect of a drug candidate on the expression of the immunogenic target in a cell line, or a cell or tissue of a patient. The expression profiling technique may be combined with high throughput screening techniques to allow rapid identification of useful compounds and monitor the effectiveness of treatment with a drug candidate (see, for example, Zlokarnik, et al., Science 279, 84-8 (1998)). Drug candidates may be chemical compounds, nucleic acids, proteins, antibodies, or derivatives therefrom, whether naturally occurring or synthetically derived. Drug candidates thus identified may be utilized, among other uses, as pharmaceutical compositions for administration to patients or for use in further screening assays.

Administration of a composition of the present invention to a host may be accomplished using any of a variety of techniques known to those of skill in the art. The composition(s) may be processed in accordance with conventional methods of pharmacy to produce medicinal agents for administration to patients, including humans and other mammals (i.e., a “pharmaceutical composition”). The pharmaceutical composition is preferably made in the form of a dosage unit containing a given amount of DNA, viral vector particles, polypeptide or peptide, for example. A suitable daily dose for a human or other mammal may vary widely depending on the condition of the patient and other factors, but, once again, can be determined using routine methods.

The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a nucleic acid or polypeptide used to induce or enhance an effective immune response. It is preferred that compositions of the present invention provide for the induction or enhancement of an anti-tumor immune response in a host which protects the host from the development of a tumor and/or allows the host to eliminate an existing tumor from the body.

For oral administration, the pharmaceutical composition may be of any of several forms including, for example, a capsule, a tablet, a suspension, or liquid, among others. Liquids may be administered by injection as a composition with suitable carriers including saline, dextrose, or water. The term parenteral as used herein includes subcutaneous, intravenous, intramuscular, intrastemal, infusion, or intraperitoneal administration. Suppositories for rectal administration of the drug can be prepared by mixing the drug with a suitable non-irritating excipient such as cocoa butter and polyethylene glycols that are solid at ordinary temperatures but liquid at the rectal temperature.

The dosage regimen for immunizing a host or otherwise treating a disorder or a disease with a composition of this invention is based on a variety of factors, including the type of disease, the age, weight, sex, medical condition of the patient, the severity of the condition, the route of administration, and the particular compound employed. For example, a poxviral vector may be administered as a composition comprising 1×10⁶ infectious particles per dose. Thus, the dosage regimen may vary widely, but can be determined routinely using standard methods.

A prime-boost regimen may also be utilized (see, for example, WO 01/30382) in which the targeted immunogen is initially administered in a priming step in one form followed by a boosting step in which the targeted immunogen is administered in another form. The form of the targeted immunogen in the priming and boosting steps are different. For instance, if the priming step utilized a nucleic acid, the boost may be administered as a peptide. Similarly, where a priming step utilized one type of recombinant virus (i.e., ALVAC), the boost step may utilize another type of virus (i.e., NYVAC). This prime-boost method of administration has been shown to induce strong immunological responses.

While the compositions of the invention can be administered as the sole active pharmaceutical agent, they can also be used in combination with one or more other compositions or agents (i.e., other immunogenic targets, co-stimulatory molecules, adjuvants). When administered as a combination, the individual components can be formulated as separate compositions administered at the same time or different times, or the components can be combined as a single composition.

Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. For instance, a viral vector such as a poxvirus may be prepared in 0.4% NaCl. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

For topical administration, a suitable topical dose of a composition may be administered one to four, and preferably two or three times daily. The dose may also be administered with intervening days during which no does is applied. Suitable compositions may comprise from 0.001% to 10% w/w, for example, from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes) and drops suitable for administration to the eye, ear, or nose.

The pharmaceutical compositions may also be prepared in a solid form (including granules, powders or suppositories). The pharmaceutical compositions may be subjected to conventional pharmaceutical operations such as sterilization and/or may contain conventional adjuvants, such as preservatives, stabilizers, wetting agents, emulsifiers, buffers etc. Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

Pharmaceutical compositions comprising a nucleic acid or polypeptide of the present invention may take any of several forms and may be administered by any of several routes. In preferred embodiments, the compositions are administered via a parenteral route (intradermal, intramuscular or subcutaneous) to induce an immune response in the host. Alternatively, the composition may be administered directly into a lymph node (intranodal) or tumor mass (i.e., intratumoral administration). For example, the dose could be administered subcutaneously at days 0, 7, and 14. Suitable methods for immunization using compositions comprising TAs are known in the art, as shown for p53 (Hollstein et al., 1991), p21-ras (Almoguera et al., 1988), HER-2 (Fendly et al., 1990), the melanoma-associated antigens (MAGE-1; MAGE-2) (van der Bruggen et al., 1991), p97 (Hu et al., 1988), and carcinoembryonic antigen (CEA) (Kantor et al., 1993; Fishbein et al., 1992; Kaufman et al., 1991), among others.

Preferred embodiments of administratable compositions include, for example, nucleic acids or polypeptides in liquid preparations such as suspensions, syrups, or elixirs. Preferred injectable preparations include, for example, nucleic acids or polypeptides suitable for parental, subcutaneous, intradermal, intramuscular or intravenous administration such as sterile suspensions or emulsions. For example, a recombinant poxvirus may be in admixture with a suitable carrier, diluent, or excipient such as sterile water, physiological saline, glucose or the like. The composition may also be provided in lyophilized form for reconstituting, for instance, in isotonic aqueous, saline buffer. In addition, the compositions can be co-administered or sequentially administered with other antineoplastic, anti-tumor or anti-cancer agents and/or with agents which reduce or alleviate ill effects of antineoplastic, anti-tumor or anti-cancer agents.

A kit comprising a composition of the present invention is also provided. The kit can include a separate container containing a suitable carrier, diluent or excipient. The kit can also include an additional anti-cancer, anti-tumor or antineoplastic agent and/or an agent that reduces or alleviates ill effects of antineoplastic, anti-tumor or anti-cancer agents for co- or sequential-administration. Additionally, the kit can include instructions for mixing or combining ingredients and/or administration.

A better understanding of the present invention and of its many advantages will be had from the following examples, given by way of illustration.

### EXAMPLES

### Example 1

### Identification of Putative MHC Binding Peptides Derived from MAGE-A1

The amino acid equence of MAGE-A1 was assessed for sequences of 9 contiguous amino acids having specific “anchor” residues, leucine (L) or methionine (M) at amino acid position #2 and leucine (1) or valine (v) at amino acid position #9,the amino-(N-) terminal being designated as position #1. Using these criteria, a number of amino acid nonamer sequences were identified including those outlined in Table III.

TABLE III

| Peptide # | Sequence | Name | SEQ. ID. NO. |
|---|---|---|---|
| 1 | KVLEYVIKV | Mage A1 278 | 1 |
| 2 | FLWGPRALA | Mage A1 264 | 5 |
| 3 | LQLVFGIDV | Mage A1 151 | 2 |
| 4 | CILESLFRA | Mage A1 92 | 6 |
| 5 | FLIIVLVMI | Mage A1 194 | 7 |
| 6 | IMPKTGFLI | Mage A1 188 | 8 |
| 7 | KVADLVGFL | Mage A1 105 | 9 |
| 8 | LVLGTLEEV | Mage A1 38 | 10 |
| 9 | KASESLQLV | Mage A1 146 | 11 |
| 10 | YVLVTCLGL | Mage A1 169 | 12 |
| 11 | YVIKVSARV | Mage A1 282 | 13 |
| 12 | RALAETSYV | Mage A1 269 | 14 |
| 13 | VITKKVADL | Mage A1 101 | 15 |
| 14 | SAYGEPRKL | Mage A1 230 | 3 |
| 15 | LIIVLVMIA | Mage A1 195 | 16 |
| 16 | GTLEEVPTA | Mage A1 41 | 17 |
| 17 | QEALGLVCV | Mage A1 20 | 18 |
| 18 | STSCILESL | Mage A1 89 | 19 |
| 19 | TKAEMLESV | Mage A1 124 | 20 |
| 20 | KTGFLIIVL | Mage A1 191 | 21 |
| 21 | LAETSYVKV | Mage A1 270 | 22 |
| 22 | SLHCKPEEA | Mage A1 7 | 23 |
| 23 | MSLEQRSL | Mage A1 1 | 24 |
| 24 | ALEAQQEAL | Mage A1 15 | 25 |
| 25 | ILESLFRAV | Mage A1 93 | 26 |
| 26 | CLGLSYDGL | Mage A1 174 | 4 |
| 27 | ALREEEEGV | Mage A1 301 | 27 |

Synthetic peptides corresponding to those listed in Table III were prepared. Solid phase peptide syntheses were conducted on an ABI 430A automated peptide synthesizer according to the manufacturer's standard protocols. The peptides were cleaved from the solid support by treatment with liquid hydrogen fluoride in the presence of thiocresole, anisole, and methyl sulfide. The crude products were extracted with trifluoroacetic acid (TFA) and precipitated with diethyl ether. All peptides were stored in lyophilized form at −20° C.

### Example 2

### Identification of Immunogenic Peptides

The HLA-A2Kb transgenic mouse model was used in order to determine which of the peptides correspond to CTL epitopes that are recognized in vivo. HLA-A2Kb mice are mice of the B1O background which are transgenic for the A2Kb chimeric gene. These mice were purchased from the Scripps Clinic in California, USA. The mice were immunized with an ALVAC vector which expresses the complete MAGE-1 coding sequence. The ALVAC vector was obtained from Virogentics, Inc., Troy, N.Y. To immunize the mice, the ALVAC MAGE-A1 vector was injected intramuscularly twice with an interval of three weeks between the first and second injection.

Three weeks following the last vector administration, spleens (3 from each group) were harvested and single cell suspensions were generated. Splenocytes were then transferred to at least 5 flasks representing one flask per group of peptides. The top 25 predicted peptides generated from the immunizing antigen were split into groups of 5 and added to each flask of splenocytes at a concentration 20 μg/peptide for a total of 100 μg. The stimulating cultures were left for 5-7 days being supplemented with fresh medium every 2 days. After this time period, only T cells which are specific for the presented peptides would be activated and the splenocyte cultures were ready to be assayed.

The splenocytes from the immunized mice were harvested from each flask by resuspending the cells vigorously followed by collection in 50 ml tubes (Falcon # 352098). The cells were centrifuged, the media discarded and the cells were washed once in Hanks Balanced salt solution (HBSS GibcoBRL # 24020-117) and resuspended in 1 ml of AIM V medium.

ELISPOT assay plates (Millipore MAHAS4510) were prepared by coating with 100 μl of anti-mouse IFN gamma (Pharmingen #554431) in 0.1M sodium hydrogen phosphate, pH 9.0 at concentration of 2 μg/ml. Plates were sealed in a plastic bag and placed at 4° C., overnight. The following day, the plates were washed 4 times with excess PBS, blocked with 300 μl of 1% BSA in PBS per well, and incubated at room temperature for at least 1 hour.

Cell counts were performed on the harvested cultured cells and a total of 10⁵ splenocytes were added per well of the prepared ELISPOT plates. To assay for specific reactivity P815A2Kb cells which had been pulsed with peptides were used as stimulators. Since P815A2Kb cells share only the A2Kb class I allele in common with the transgenic mice this allows one to specifically identify only A2Kb binding peptides.

The stimulator cells were prepared as follows. Fifty micrograms of any given individual peptide was pulsed onto 10⁶ P815A2Kb cells for 3 hours at 37° C. The pulsed cells were then irradiated at 12000-15000 rads to prevent overgrowth in the ELISPOT wells.

To each test well of the ELISPOT plate, 10⁵ pulsed P815A2Kb cells were added. Control wells were setup with irradiated unpulsed P815A2Kb cells as well as P815A2Kb cells pulsed with an irrelevant (not derived from the immunizing antigen) HLA-A0201 binding peptide. To measure the total number of T cells capable of responding in culture, PMA and ionomycin control wells were included in each assay.

The assay plates were then incubated overnight at 37° C. in 5% carbon dioxide. The next day the plates were washed in deionized water and a mix of PBS/Tween 20 to remove the cells. IFN gamma which had been secreted from activated T cells and which had bound to the anti-mouse IFN gamma coated on the bottom of each well was detected using biotinylated anti-mouse IFN gamma (Pharmingen # 554410). This antibody was incubated for 3 hours at room temperature to allow for binding to the IFN gamma. The plates were then washed as described above and the alkaline phosphatase conjugate (Extravidin Sigma #E2636) was added for 1 hour at room temperature. The unbound enzyme was then removed from the plate with vigorous washing and the enzyme substrate added (Sigma #B5655) in the dark, and allowed to develop until the IFN gamma spots were visible.

Repetitions of this assay revealed four immunogenic peptides that were capable of eliciting epitope-specific IFN γ responses in the spleens of mice immunized with ALVAC MAGE-A1. IFNγ responses were elicited by four of the MAGE-A1 peptides (Table IV). The control EBV peptide and the antigen presenting cells alone or the spleenocytes alone did not elicit IFNγ responses.

TABLE IV
MAGE-A1 Peptides

| | | |
|---|---|---|
| MAGE-A1 278: | KVLEYVIKV | (SEQ ID NO: 1) |
| MAGE-A1 151: | LQLVFGIDV | (SEQ ID NO: 2) |
| MAGE-A1 230: | SAYGEPRKL | (SEQ ID NO: 3) |
| MAGE-A1 174: | CLGLSYDGL | (SEQ ID NO: 4) |

Exemplary nucleic acid sequencse coding for the immunogenic MAGE-A1 peptides (i.e. SEQ ID.NOS:1-4) were deduced by reference to the known MAGE-1A nucleic acid sequence, as shown below:

| | |
|---|---|
| Peptide | Nucleic Acid Sequence |

| | |
|---|---|
| MAGE-A1 278 | (SEQ.ID.NO.28) |

AAAGTCCTTGAGTATGTGATCAAGGTC

| | |
|---|---|
| MAGE-A1 151 | (SEQ.ID.NO.29) |

TTGCAGCTGGTCTTTGGCATTGACGTG

| | |
|---|---|
| MAGE-A1 230 | (SEQ.ID.NO.30) |

AGTGCCTATGGGGAGCCCAGGAAGCTG

| | |
|---|---|
| MAGE-A1 174 | (SEQ.ID.NO.31) |

TGCCTAGGTCTCTCCTATGATGGCCTG

Other nucleic acid sequences may encode the MAGE-A1 peptides as well. Table V illustrates the various the codons that could be utilized to encode MAGE-A1 278, 151, 230, and 174 in a nucleic acid sequence. The codons coding for the various amino acid sequences may be used in any combination to encode the MAGE peptides.

TABLE V

| 278 | K | V | L | E | Y | V | I | K | V |
|---|---|---|---|---|---|---|---|---|---|
| | AAA | GTT | CTT | GAA | TAT | GTT | ATT | AAA | GTT |
| | AAG | GTC | CTC | GAG | TAC | GTC | ATC | AAG | GTC |
| | | GTA | CTA | | | GTA | ATA | | GTA |
| | | GTG | CTG | | | GTG | | | GTG |
| 151 | L | Q | L | V | F | G | I | D | V |
| | CTT | CAA | CTT | GTT | TTT | GGT | ATT | GAT | GTT |
| | CTC | CAG | CTC | GTC | TTC | GGC | ATC | GAC | GTC |
| | CTA | | CTA | GTA | | GGA | ATA | | GTA |
| | CTG | | CTG | GTG | | GGG | | | GTG |
| 230 | S | A | Y | G | E | P | R | K | L |
| | TCT | GCT | TAT | GGT | GAA | CCT | AGA | AAA | CTT |
| | TCC | GCC | TAC | GGC | GAG | CCC | AGG | AAG | CTC |
| | TCA | GCA | | GGA | | CCA | | | CTA |
| | TCG | GCG | | GGG | | CCG | | | CTG |
| 174 | C | L | G | L | S | V | D | G | L |
| | TGT | CTT | GGT | CTT | TCT | TAT | GAT | GGT | CTT |
| | TGC | CTC | GGC | CTC | TCC | TAC | GAC | GGC | CTC |
| | | CTA | GGA | CTA | TCA | | | GGA | CTA |
| | | CTG | GGG | CTG | TCG | | | GGG | CTG |

### Example 3

### HLA-A0201 Binding of MAGE-A1 Derived Peptides

The ability of the MAGE-A1 derived peptides to be presented in the context of HLA-A2 was determined by the ability of such peptides stabilize membrane-bound HLA-A0201 molecules The T2 cell line (Dr. Peter Creswell, Yale University) has been well documented to have a defective TAP (e.g. Transporter for Antigen Processing) transporter function. As a result, the majority of intracellularly generated peptides are not transported into the endoplasmic reticulum and thus are unable to associate with newly synthesized HLA class 1 MHC molecules (e.g. HLA-A0201; Salter, R D and Creswell, P. (1986) EMBO J 5:943). The majority of the HLA-A0201 molecules displayed on the surface of T2 cells are therefore empty (contain no peptides) and thus are unstable. The stability of the surface HLA-A0201 molecules can be restored upon interaction with suitable exogenous peptides. The stabilization of the conformation of the class 1 MHC molecules is accompanied by the formation of an immunodominant epitope recognized by a mouse monoclonal antibody (designated BB7.2; American Type Culture Collection (ATCC)). Thus, the detection of this specific epitope is indicative of stable membrane-bound HLA-A0201 molecules loaded with peptide which, in turn, demonstrates that such peptide is capable of presentation by HLA-10201. Subsequent dissociation of peptides from the HLA class 1 MHC molecules results in the loss of BB7.2 monoclonal antibody binding.

In order to determine whether the identified nonapeptides can be presented by HLA-A0201, T2 cells were propagated in RPMI complete medium (RPMI medium supplemented with 10% heat-inactivated bovine serum, 120.0 units per ml of penicillin G sodium, 120 μg per ml of streptomycin sulphate, and 0.35 mg per ml of L-glutamine). The ability of MAGE-A1 derived peptides to bind and stabilize surface HLA-A0201 molecules on T2 cells was determined utilizing a standard protocol (Deng, Y. (1997) J Immunol 158:1507-1515). The required number of T2 flasks were incubated overnight at 26° C. serum-free culture medium (RPMI medium supplemented with 120.0 units per ml of penicillin G sodium and 0.35 mg per ml of L-glutamine). The next day, cells were washed with RPMI medium (without bovine serum) and then resuspended in denaturing solution (300 mM Glycine in 1% BSA, pH 2.5) for 3 min, in order to strip the existing HLA A2 molecules of endogenous peptide. The stripped T2 cells were washed at once in an excess of RPMI media (without bovine serum) to neutralize the acidic stripping solution. To load the peptide of interest into the HLA-A2 peptide binding groove, 20 μg of specific peptide was pulsed onto 10⁶ denatured T2 cells in 2 ml peptide loading media (RPMI medium supplemented with 120.0 units per ml of penicillin G sodium; 0.35 mg per ml of L-glutamine; 1× sodium pyruvate; 1× non-essential amino acids; 1×2-mercapto-ethanol) for 4 hours at 26° C. The cells were washed in cold 1% BSA in PBS and resuspended in 100 μl of cold 1% BSA in PBS to prevent MHC protein turn over. To detect the stabilization of the HLA-A2 molecules, 5.0 μg of monoclonal antibody BB7.2 was added to each test sample. The reaction was allowed to proceed on ice for 30 min. The cells were washed once with 15 ml cold BSA/PBS and resuspended in 100 μl of cold BSA/PBS. The binding of BB7.2 was detected via the addition of 1.0 μg per test of goat anti-mouse IgG-Fc fluorescein (FITC) conjugate (BETHYL Laboratories Inc). After a 30 min incubation on ice, cells were washed once with 15 ml cold BSA/PBS and resuspended in 1 ml of cold BSA/PBS. The samples were then analyzed by Flow Cytometry, and the results were expressed in units of Fluorescence Index (FI), calculated by the equation:

$$\frac{\begin{matrix}Mean Fluorescence ( MF ) of peptide \text{-} treated sample - \\ MF of control sample\end{matrix}}{MF of control sample ( cells not peptide treated )}$$

The HLA binding activity of the four peptides according to SEQ. ID. NOS. 1-4 are shown in Table VI.

TABLE VI
Binding and Stabilization of MAGE-A1 derived peptides to T2 cells

| | | | Fluorescence |
|---|---|---|---|
| Peptide | Peptide Sequence | SEQ ID NO. | Index (FI) |
| MAGE-A1 278 | KVLEYVIKV | 1 | 2.18 |
| MAGE-A1 151 | LQLVFGIDV | 2 | 0.52 |
| MAGE-A1 230 | SAYGEPRKL | 3 | 1.61 |
| MAGE-A1 174 | CLGLSYDGL | 4 | 1.73 |
| Background Control | | | 0 |

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. An immunogenic peptide selected from the group consisting of SEQ ID NO.:1, SEQ ID NO.:2, SEQ ID NO.:3, and SEQ ID NO.:4.

2. An immunogenic composition comprising a peptide of claim 1 in a pharmaceutically acceptable carrier.

3. An immunogenic polypeptide comprising an amino acid sequence selected from the group consisting of SEQ ID NO.:1, SEQ ID NO.:2, SEQ ID NO.:3, and SEQ ID NO.:4.

4. An immunogenic composition comprising a polypeptide of claim 3 in a pharmaceutically acceptable carrier.

5. An isolated nucleic acid encoding an immunogenic peptide selected from the group consisting of SEQ ID NO.:1, SEQ ID NO.:2, SEQ ID NO.:3, and SEQ ID NO.:4.

6. A composition comprising a nucleic acid of claim 5 in a pharmaceutically acceptable carrier.

7. An isolated nucleic acid encoding an immunogenic peptide selected from the group consisting of:

| | |
|---|---|
| AAAGTCCTTGAGTATGTGATCAAGGTC; | (SEQ.ID.NO.28) |
| TTGCAGCTGGTCTTTGGCATTGACGTG; | (SEQ.ID.NO.29) |
| AGTGCCTATGGGGAGCCCAGGAAGCTG; and, | (SEQ.ID.NO.30) |
| TGCCTAGGTCTCTCCTATGATGGCCTG. | (SEQ.ID.NO.31) |

8. A composition comprising a nucleic acid of claim 7 in a pharmaceutically acceptable carrier.

9. An expression vector comprising a nucleic acid sequence encoding an immunogenic peptide selected from the group consisting of SEQ ID NO.: 1, SEQ ID NO.:2, SEQ ID NO.:3, and SEQ ID NO.:4.

10. The expression vector of claim 9 wherein the vector is a plasmid or a viral vector.

11. The expression vector of claim 10 wherein the viral vector is selected from the group consisting of poxvirus, adenovirus, retrovirus, herpesvirus, and adeno-associated virus.

12. The expression vector of claim 11 wherein the viral vector is a poxvirus selected from the group consisting of vaccinia, NYVAC, avipox, canarypox, ALVAC, ALVAC(2), fowlpox, and TROVAC.

13. The expression vector of claim 12 wherein the viral vector is a poxvirus selected from the group consisting of NYVAC, ALVAC, and ALVAC(2).

14. An expression vector comprising a nucleic acid sequence encoding an immunogenic peptide selected from the group consisting of:

| | |
|---|---|
| AAAGTCCTTGAGTATGTGATCAAGGTC; | (SEQ.ID.NO.28) |
| TTGCAGCTGGTCTTTGGCATTGACGTG; | (SEQ.ID.NO.29) |
| AGTGCCTATGGGGAGCCCAGGAAGCTG; and, | (SEQ.ID.NO.30) |
| TGCCTAGGTCTCTCCTATGATGGCCTG. | (SEQ.ID.NO.31) |

15. The expression vector of claim 14 wherein the vector is a plasmid or a viral vector.

16. The expression vector of claim 15 wherein the viral vector is selected from the group consisting of poxvirus, adenovirus, retrovirus, herpesvirus, and adeno-associated virus.

17. The expression vector of claim 16 wherein the viral vector is a poxvirus selected from the group consisting of vaccinia, NYVAC, avipox, canarypox, ALVAC, ALVAC(2), fowlpox, and TROVAC.

18. The expression vector of claim 17 wherein the viral vector is a poxvirus selected from the group consisting of NYVAC, ALVAC, and ALVAC(2).

19. A composition comprising an expression vector of claim 7 or 14 in a pharmaceutically acceptable carrier.

20. A method for preventing or treating cancer comprising administering to a host a peptide of claim 1.

21. A method for preventing or treating cancer comprising administering to a host a polypeptide of claim 3.

22. A method for preventing or treating cancer comprising administering to a host an expression vector of claim 7.

23. A method for preventing or treating cancer comprising administering to a host an expression of claim 14.

24. A host cell comprising a peptide selected from the group consisting of SEQ ID NO.:1, SEQ ID NO.:2, SEQ ID NO.:3, and SEQ ID NO.:4.

25. A method for preventing or treating cancer comprising administering to a patient a host cell of claim 23.

26. A method for inducing an immune response in a patient comprising:
a) obtaining autologous cells from said patient;
b) pulsing said cells in vitro with a peptide selected from the group consisting of SEQ ID NO.:1, SEQ ID NO.:2, SEQ ID NO.:3, and SEQ ID NO.:4; and,
c) administering the cells of step b to said patient.

27. A method for inducing an immune response in a patient comprising:
a) obtaining autologous cells from said patient;
b) transfecting said cells in vitro with a nucleic acid encoding a peptide selected from the group consisting of SEQ ID NO.: 1, SEQ ID NO.:2, SEQ ID NO.:3, and SEQ ID NO.:4; and,
c) administering the cells of step b to said patient.
